(19) United States
(12) Patent Application Publication
(10) Pub. No.: US 2005/0215920 A1
(43) Pub. Date: Sep. 29, 2005

(54) Endocervical curettings receiver

(76) Inventor: Nessim N. Isa, Saint John (CA)

Correspondence Address: MARKS & CLERK, 1075 NORTH SERVICE ROAD WEST, SUITE 203, OAKVILLE, ON, L6M 2G2, CA

(21) Appl. No.: 10/810,560

(22) Filed: Mar. 29, 2004

(51) Int. Cl. A61B 10/00

(52) U.S. Cl. 600/562; 600/564; 600/570

(57) ABSTRACT

An endocervical curettings receiver is provided for facilitate the collection of substantially all of the tissue samples taken during a endocervical curetting procedure. This allows all of the tissue samples taken to be analysed. The endocervical curettings receiver has a handle to which is connected a collector have a truncated, spoon shaped appearance, and having smooth edges around the circumference of the collector. An improved device and method for the collection of substantially all of the tissue samples taken is provide.

### FIELD OF THE INVENTION

This invention relates to surgical instruments used for collecting the tissues from endocervical scrapings. More particularly, it is concerned with collection of tissue specimens which are generated in endocervical curetting procedures. Such specimens are needed for biopsies when abnormal PAP smear results are obtained.

### BACKGROUND OF THE INVENTION

During the 1940's, Dr. G. Papanicolaou developed a screening test which has become the most widely used screening technique for detecting abnormal cervical cells. Today, this test is more commonly known as the PAP test or the PAP smear test. Typically, the PAP test is performed in the physician's office as part of a woman's routine gynecological examination. The test involves collecting cervical cells via a brush, stick or swab that is used to loosen and then collect cells that can be examined microscopically.

If the results of the PAP test are abnormal, this could be an indication of a series of potential health issues ranging from precancerous atypical dysplasia to invasive cancer, and in particular, cancer of the cervix. Once an abnormal PAP test has occurred, the physician will routinely conduct a colposcopy evaluation in which the cervix is viewed under microscope after the local area of the cervix has been treated with an acidic solution. At the same time, the physician commonly takes tissue specimens from the area of the cervix, using an endocervical curetting apparatus, so that the specimens may be subsequently tested for the presence of, inter alia, abnormal tissue cells.

However, the colposcopy is typically useful only for observing the cervix opening and surrounding area. Depending on the type and size of any abnormalities observed, the physician will collect tissue samples using an endocervical curette. Further, the colposcopy procedure may not provide a clear view of all of the cervix. As such, the physician will likely take random tissue samples from other areas of the cervix where it is difficult to see the tissue.

The endocervical curetting instrument is commonly a small, spoon shaped device having sharp edges for cutting and/or scrapping of tissues from the cervical walls. Commonly used types of endocervical curettes are described in, for example, U.S. Pat. Nos. 4,641,662, 4,932,957 and 5348023. These devices are all useful for scrapping and/or cutting tissue (or the “endocervical curettings”) from the cervical area. The collect tissue is then typically trapped in the “basket” of the curette (e.g. two bands of steel across the bottom of the curette). Once in the basket, the tissue specimen is removed while being physically attached to the curette. Alternatively, the tissue or any fluid samples could be collected using suction. However, given the size and shape of the curette, it is frequently not possible to collect all of the tissue specimens which have been cut or scrapped from the cervix. Also, suction of the tissue specimens results in loss of material which becomes stuck to the walls of the suction equipment.

Human papillomavirus (HPV) has been scientifically documented to cause a large majority of all cervical cancers, and testing of HPV can accurately identify many precancerous changes in the cervix. Since the present procedures employed to obtain endocervical curettings during colposcopy evaluation can result in the loss of a considerable amount of the necessary tissue sample, the accuracy of identifying pre-malignant cells may be affected. This could even result in missing the diagnosis of cervical cancer. As such, it is important that all tissue specimens generated during the endocervical curetting procedure be collected for testing.

To overcome these difficulties, it would be advantageous to provide a device which would assist in the collection of endocervical curettings, and in particular, provide a device which would assist in ensuring that essentially all of the endocervical curettings were collected for testing.

### SUMMARY OF THE INVENTION

Accordingly, it is a principal advantage of the present invention to be able to provide a endocervical curettings receiver which is adapted to be used to assist in collecting endocervical curettings.

It is a further advantage of the present invention to be able to provide an endocervical curettings receiver which can be positioned near the cervix, and in which, endocervical curettings can be deposited as they are scrapped and/or cut from the cervix area.

The advantages set out hereinabove, as well as other objects and goals inherent thereto, are at least partially or fully provided by the endocervical curettings receiver of the present invention, as set out herein below.

Accordingly, in one aspect, the present invention provides an endocervical curettings receiver comprising an elongate handle having a longitudinal axis;
collection means for retaining said endocervical curettings, attached to said handle, at a proximate end of said collection means, wherein said collection means preferably has a concave, spoon shape, and has a truncated end at a distal end of said collection means; and
said collection means has a circumferential edge which is smooth to avoid cutting or scrapping of the cervical area.

In a further aspect, the present invention also provides a method for obtaining tissue from inside a living body, the method comprising the steps of:
inserting a curette having sharp cutting edges inside a body,
inserting the collection means of an endocervical curettings receiver inside the body, and positioning said receiver adjacent to said curette, wherein said receiver comprises an elongate handle having a longitudinal axis; collection means for retaining said endocervical curettings, attached to said handle, at a proximate end of said collection means, wherein said collection means preferably has a concave, spoon shape, and has a truncated end at a distal end of said collection means; and said collection means has a circumferential edge which is smooth to avoid cutting or scrapping of the cervical area;
moving said inserted curette to cause one or more cutting edges on said curette to cut tissue from the body;
placing or collecting said cut tissue within the collection means; and
withdrawing said curette and said collection means from the body.

A key feature of the endocervical curettings receiver of the present invention is that it is somewhat larger than the curette since it is not expected that the receiver will need to be placed within the cervix, per se. Instead, the receiver is positioned outside of the cervix but in a convenient location for receiving any tissue samples collected from the cervix area using the curette.

A second feature of the endocervical curettings receiver of the present invention is that it does not have sharp edges, and therefore, it is not used for scrapping or cutting of tissue samples. It can therefore be placed adjacent to the cervix to collect the tissue samples generated by use of the curette.

### BRIEF DESCRIPTION OF THE DRAWINGS

Embodiments of this invention will now be described by way of example only in association with the accompanying drawings in which:

FIG. 1 is a perspective view of the endocervical curettings receiver of the present invention; and

FIG. 2 is a top view thereof.

### DETAILED DESCRIPTION OF THE PREFERRED EMBODIMENTS

The novel features which are believed to be characteristic of the present invention, as to its structure, organization, use and method of operation, together with further objectives and advantages thereof, will be better understood from the following drawings in which a presently preferred embodiment of the invention will now be illustrated by way of example only. In the drawings, like reference numerals depict like elements.

It is expressly understood, however, that the drawings are for the purpose of illustration and description only and are not intended as a definition of the limits of the invention.

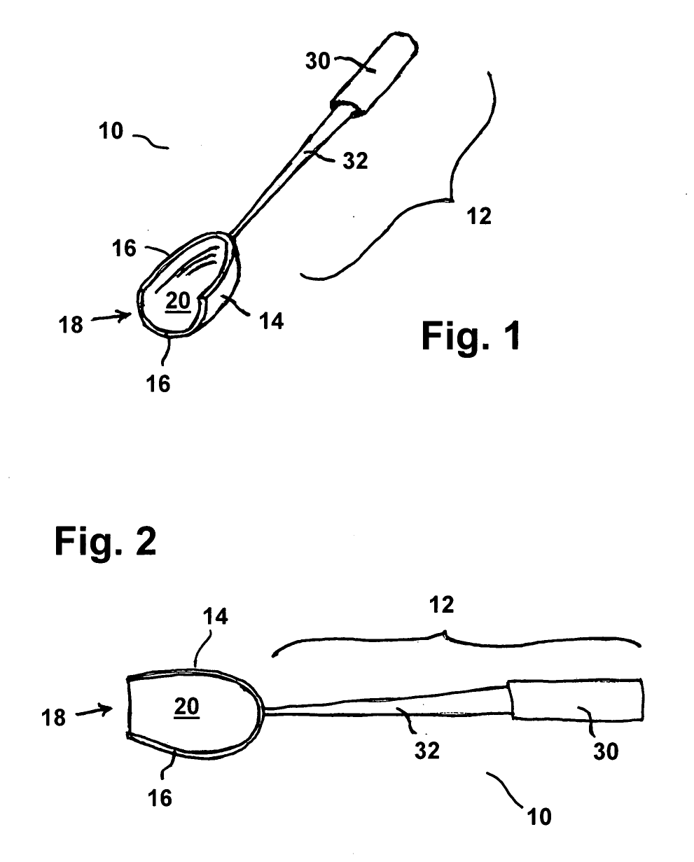

Referring to FIGS. 1 and 2, an endocervical curettings receiver 10 is shown having an elongated handle 12 which handle is shown having a single longitudinal axis.

An upper grip portion 30 of handle 12 is slightly larger than the lower half 32 of the handle, providing a more comfortable and controllable gripping surface with which the user holds the instrument. The lower section 32 of handle 12 is tapered so that it decreases in cross-section diameter as it moves away from grip portion 30. It will be apparent that other handle designs might also be used, however, this handle design provides comfortable, ergonomic control of the instrument while being simple and inexpensive to manufacture. Also, because the instrument as shown in this embodiment is symmetrical, either the left or the right hand many be used.

At one end of handle 12 is a concave, truncated spoon shaped collector 14, having an edge 16 which extends around the circumference of collector 14. Edge 16 is smooth so as to avoid cutting and/or scraping any surrounding tissue.

Collector 14 has a truncated end 18, and a collection zone 20 within its concave portion.

The receiver might be made of any material suitable for insertion into a living body. However, preferred materials would be stainless steel, or the like, which can be sterilized between uses. Alternatively, the receiver might be made partially or completely of plastic or other materials which can be discarded after use.

Receiver 10 is approximately 25 cm in length. However, its length can be varied depending on the preferences of the user. Typically, the length will be between 20 and 30 cm.

The overall size of collector 14 can vary but it is to be kept to a size suitable for placement within the body. Preferably, it has a length of between 1 and 5 cm, and more preferably, a length of between 2 and 3 cm. A preferred with is between 1 and 4 cm, and more preferably, a width of between 2 and 3 cm. A preferred depth of the collection zone is between 1 and 4 cm, with a preferred depth of between 1.5 and 3 cm.

It should be noted that the endocervical curettings receiver of the present invention can be many times larger than the curette since it is not necessary for the received to enter the cervix. As such, the collector of the receiver can commonly be at least twice, and more preferably between 5 and 20 times the size (in volume) of a collector section formed as part of the curette.

In operation, collector 14 of receiver 10 is inserted into the body, and positioned adjacent to the cervix. If desired, truncated end 18 can rest against the body. A curette is used to cut and/or scrap tissue samples from the body in the area of the cervix. The tissue samples cut and/or scrapped by the curette are deposited into collection zone 20. At the end of the procedure, collector 14 is removed from the body, and the tissue samples collected therein can be analysed.

Using this procedure, it is not necessary to continually remove the curette from the body in order to collect the tissue samples. Further, given the larger size of the receiver, more material can be collected than in the relatively small collection areas provided on some curettes. As a result, substantially all of the curetted material can be collected and retained within the collection zone of the receiver, and very little of the curetted material is lost during the procedure.

It is expected that the receiver of the present invention can be used during colposcopy procedures where endocervical curettings are to be collected. However, it might also be used during any other curetting procedure such as diagnostic dilation and curettage, or the like.

Thus, it is apparent that there has been provided, in accordance with the present invention, an endocervical curettings receiver which fully satisfies the goals, objects, and advantages set forth hereinbefore. Therefore, having described specific embodiments of the present invention, it will be understood that alternatives, modifications and variations thereof may be suggested to those skilled in the art, and that it is intended that the present specification embrace all such alternatives, modifications and variations as fall within the scope of the appended claims.

Additionally, for clarity and unless otherwise stated, the word “comprise” and variations of the word such as “comprising” and “comprises”, when used in the description and claims of the present specification, is not intended to exclude other additives, components, integers or steps.

Moreover, the words “substantially” or “essentially”, when used with an adjective or adverb is intended to enhance the scope of the particular characteristic; e.g., substantially planar is intended to mean planar, nearly planar and/or exhibiting characteristics associated with a planar element.

Further, use of the terms “he”, “him”, or “his”, is not intended to be specifically directed to persons of the masculine gender, and could easily be read as “she”, “her”, or “hers”, respectively.

Also, while this discussion has addressed prior art known to the inventor, it is not an admission that all art discussed is citable against the present application.

1. An endocervical curettings receiver comprising:
an elongate handle having a longitudinal axis;
collection means for collecting and retaining endocervical curettings, attached to said handle, at a proximate end of said collection means; and
wherein said collection means has a circumferential edge which is smooth to avoid cutting or scrapping of the cervical area.

2. An endocervical curettings receiver as claimed in claim 1 wherein said collection means has a concave, spoon shape.

3. An endocervical curettings receiver as claimed in claim 2 wherein said collection means has a truncated end at a distal end of said collection means.

4. An endocervical curettings receiver as claimed in claim 1 wherein said receiver is fabricated from stainless steel.

5. An endocervical curettings receiver as claimed in claim 1 wherein at least part of said receiver is fabricated from a plastic material that can be discarded after use.

6. A method for obtaining tissue from inside a living body, the method comprising the steps of:
inserting a curette having sharp cutting edges inside a body,
inserting the collection means of an endocervical curettings receiver inside the body, and positioning said receiver adjacent to said curette, wherein said receiver comprises an elongate handle having a longitudinal axis; collection means for retaining said endocervical curettings, attached to said handle, at a proximate end of said collection means, wherein said collection means preferably has a concave, spoon shape, and has a truncated end at a distal end of said collection means; and said collection means has a circumferential edge which is smooth to avoid cutting or scrapping of the cervical area;
moving said inserted curette to cause one or more cutting edges on said curette to cut tissue from the body;
placing or collecting said cut tissue within the collection means; and
withdrawing said curette and said collection means from the body.

7. A method as claimed in claim 6 wherein said collector of said endocervical curettings receiver is at least twice the size (in volume), of a collector section formed as part of the curette.

8. A method as claimed in claim 7 wherein said collector is between 5 and 20 times the size (in volume), of a collector section formed as part of the curette.
